(19) United States
(12) Patent Application Publication
(10) Pub. No.: US 2009/0275658 A1
(43) Pub. Date: Nov. 5, 2009

(54) Method for Producing a Raw Oil from Mixtures of Micro-Organisms and Plants, Oil Produced According to Said Method and Specific Uses of the Thus Produced Oil and, Optionally, Additional Refined Oil

(76) Inventor: Dirk Fabritius, Ansbach (DE)

Correspondence Address: HAMMER & ASSOCIATES, P.C., 3125 SPRINGBANK LANE, SUITE G, CHARLOTTE, NC, 28226, US

(21) Appl. No.: 11/720,990

(22) PCT Filed: Dec. 20, 2005
(86) PCT No.: PCT/EP2005/013688

§ 371 Date: Jul. 31, 2007

(30) Foreign Application Priority Data
Dec. 23, 2004 (DE) 10 2004 062 141.1

(51) Int. Cl. A61K 31/20; C12P 7/64

(52) U.S. Cl. 514/560; 435/134

(57) ABSTRACT

The invention relates to a method for producing a raw oil from micro-organisms and plants, oil produced according to said method and specific uses of the thus produced oil and, optionally, additional refined oil in foodstuffs, in food supplements, or in cosmetic or pharmaceutical compositions.

A method for producing a raw oil from mixtures of micro-organisms and plants, the oil thus produced, as well as the specific uses of the oil that has been produced thus and, optionally, additionally refined.

The present invention relates to a method for producing a raw oil from mixtures of micro-organisms and plants, the oil thus produced, as well as the specific uses of the oil that has been produced thus and, optionally, additionally refined, in foodstuffs, in food supplements, or in cosmetic or pharmaceutical compositions.

Oils which contain long-chain polyunsaturated fatty acids such as for example arachidonic acid (ARA), docosapentaenoic acid (DPA), docosahexaenoic acid (DHA), eicosapentaenoic acid (EPA), stearidonic acid (SA) or di-homo-gamma linolenic acid (DHGLA), can be obtained from the cultivation of micro-organisms. For this, usually one micro-organism is cultivated that is rich in one or more of the aforementioned fatty acids, the biomass is harvested from the culture, is digested, and the oil is isolated. To isolate the oil from the biomass, until now the methods that have been used are above all extraction methods with organic solvents, for example hexane, or with supercritical liquids. In general, the oil is extracted from the biomass by percolation of the dried biomass with hexane. Such processes of extraction with organic solvents are described for example in WO 97/37032, WO 97/43362, EP 515460 and many other documents. A particularly detailed description is also to be found in the Journal of Dispersion Science and Technology, 10, 561-579, 1989, “Biotechnological Processes for the Production of PUFAs”.

The usual objective is to achieve as high a proportion of triglycerides in the finished oil as possible, in particular if it is to be used for foods or for pharmaceutical/cosmetic purposes. Phospholipids and free fatty acids are regarded as impurities in the finished oil. Likewise, solvents should no longer be present in the finished oil in any significant amounts. When using a biomass with a high phospholipid content, it is possible to extract the phospholipids if necessary without the addition of further solvents (e.g. ethanol), with the aid of the triglycerides that are naturally present, which also act as solvents. In particular, phospholipids containing PUFAs are difficult to isolate, expensive, but desirable for many applications.

For this reason, the extraction of PUFAs with solvents has a number of fundamental disadvantages. Thus for example during extraction, the PUFAs can react with hot solvents, or with atmospheric oxygen during distilling, and thus form specific decomposition products (degradation), which are highly undesirable, through unwanted oxidation, on the double bond. Furthermore, if the solvent is to be completely separated off, this generally requires heat treatment at high temperature, and these conditions further benefit degradation. Where organic solvents are used, there is always a risk of explosion, which makes expensive safety precautions necessary. Hexane is under discussion as a neurotoxin. There are therefore statutory limit values that have to be monitored, which increases costs further. The disposal of the corresponding waste is also cost-intensive.

Furthermore, the solvent, such as for example hexane, can also release other constituents from the biomass which are not triglycerides, and therefore represent impurities which later on either cannot be separated off at all, or only at great expense.

The raw oil that is obtained after the solvent has been separated off therefore then needs to be further refined if the oil is intended for use as edible oil and/or for pharmaceutical purposes. The refining steps include de-gumming (de-sliming), neutralisation with alkaline solution, de-colourisation, de-waxing and deodorisation, in order to at least partially remove the impurities. This however also means that the oil that contains the highly unsaturated fatty acids is exposed to conditions in which the occurrence of physico-chemical reactions in the unsaturated fatty acids becomes probable.

There are however also methods for the extraction of biomasses in which no solvent is used; see e.g. EP-A-1178118. According to the method described there, the use of a solvent is avoided in that an aqueous suspension of the biomass is produced, and the oil phase is separated off from the aqueous phase through centrifuging. The aqueous phase contains cell wall debris and a certain amount of water-soluble material which originates from the biomass. A great disadvantage of this method is the fact that the raw oil contains numerous impurities, such as for example polar lipids, protein residues and so on. The raw oil obtained in this way must therefore be refined using conventional methods such as have been previously described for vegetable oils and microbial oils.

In WO 2004/022678, a method is described for producing microbial oils that contain PUFAs, in which an oil that contains PUFA is obtained from a biomass of micro-organisms, through pressing. The remaining press cake is mixed with a carrier oil, and pressed again, wherein the carrier oil is intended to help to release any remaining PUFAs from the press cake, wherein one obtains a second press oil which has a lower PUFA concentration than the first press oil. By mixing the two oils in variable weight proportions, PUFA concentrations can be obtained that are adapted for the respective desired application. In principle, a second oil is thus used as a solvent here. In principle, this method suffers the same disadvantages as apply to the prior art described above.

Overall, the pressing of certain biomasses, in particular of micro-organisms such as e.g. Ulkenia spec., is difficult and often requires drastic conditions such as high pressures and high temperatures. This is at the expense of yield and quality.

Furthermore, this results in a considerable nuisance due to bad smells, which also represents a hazard to personnel in the production plant.

Furthermore, the carrier oil itself can be oxidised, through which the quality would suffer. In the worst case, the carrier oil can ignite itself through oxidation (a known problem when PUFAs are stored exposed to air on large surfaces for longer periods of time (e.g. activated carbon, clay)).

The objective of the present invention is therefore to avoid the disadvantages of the prior art, in that a method for producing a stable oil is provided, wherein the oil contains one or more polyunsaturated fatty acids derived from the biomass and present in the form of triacylglycerides or phospholipids in a relatively pure state and with a high yield, and in which the oil is subject to only minimal degradation.

If the oil is to be used as an edible oil, a high proportion of triglycerides is preferred.

These as well as other objectives which, to the person skilled in the art, clearly result from the disadvantages of the prior art as discussed, are achieved through a method with all the features of claim 1.

Preferred design examples of the present invention are described and claimed in the dependent subordinate claims and the co-ordinate claims.

The method is characterised in that one or more biomasses obtained from the culture of micro-organisms, in particular from the culture of a fungus or a micro-alga, and which contain at least one of the unsaturated fatty acids ARA, DHGLA, DPA, DHA or EPA, are mixed with a second, different biomass, and that an oil is obtained by pressing the two biomasses.

For the purposes of the present invention, an oil is also to be understood as a lipid or a fat.

From the raw oil that is obtained, using process steps that are readily familiar to the person skilled in the art, individual constituents such as triglycerides or phospholipids can be purified, depending on the desired application.

The present invention thus relates to both to the raw oil that can be obtained with the method according to this invention and to the individual fractions that are also obtainable, such as for example triglyceride mixtures or phospholipid fractions.

According to a preferred design example of the present invention, the second biomass represents sunflower seeds.

The second biomass can however itself also be a microbial biomass.

According to a further preferred aspect, the microbial biomass is dried before the second biomass is mixed into it. According to a further preferred aspect, the microbial biomasses are obtained from the cultivation of the following organisms: Mortierella, Crypthecodinium (Dinoflagellates), Thraustochytrium, Schizochytrium (Labyrinthulomycetes), Phaeodactylum, Nanochlioropsis, Euglena, Tetrahymena, Spirulina as well as preferably Ulkenia spec.

It has been shown that when sunflower seeds are used, the pressing process is considerably simplified. This is due, for one thing, to the fact that the sunflower seeds serve as additional frictional agents which facilitate the digestion of the biomass. Moreover, sunflower seeds themselves are rich in high-quality vegetable oil. This is released in the course of the pressing procedure, and serves on the one hand as a lubricant for the mill, thus facilitating pressing, and the sunflower oil that is also released serves simultaneously as a solvent for the high proportions of polyunsaturated fatty acids which are present as triglycerides in the microbial biomass.

Advantageously, antioxidants in the seeds/biomass can have an additional stabilising influence on the product. Thus for example Crypthecodinium forms antioxidants that can strongly stabilise DHA oil. Thus in a quite surprising way, the method offers a simple approach for making available microbial oils that are rich in polyunsaturated fatty acids, in a gentle manner and with an excellent yield.

At the same time, by adjusting the amount of sunflower seeds that are added, oil can be obtained that contains different concentrations of PUFAs, and at the same time also contains different quantities of sunflower oil proportions. Designer oils can thus be developed that are adapted specifically to particular applications.

It is also possible to use for example as the second biomass e.g. the fungus Mortierella, which contains high proportions of ARA (arachidonic acid). When using e.g. Ulkenia as the first biomass, one obtains an oil that contains DHA and ARA. Furthermore, the proportions can be varied in that the relative proportions of the biomasses to one another are varied. If sunflower seeds or another oil plant are used as the additional biomass, the antioxidants that are contained in the plant oils have a favourable effect on the storage stability of the oils that contain PUFAs.

The present invention thus also relates to oils that can be produced with the aid of the method according to this invention, as well as to their use in foodstuffs, food supplements, pharmaceutical and cosmetic products.

According to this invention, these oils thus originate from one or more microbial biomasses, which ideally, before pressing, are mixed and/or ground with oil seeds, preferably sunflower seeds.

The production of the oil is therefore carried out with direct pressing of the biomass mixture. If necessary, this oil that is obtained then undergoes physical refining in order to obtain the desired oil.

For the purposes of the present invention, physical refining means that the result is a reduction of phospholipids and free fatty acids, and it is understood as a de-sliming treatment without the use of acids and without neutralisation. Thus it has been observed when using a biomass that contains ARA that the oil was practically completely free of fatty acids and phospholipids, and that therefore, depending on the degree of purity required, a de-sliming treatment was not necessary.

If however a biomass has been used which contained DHA, de-sliming treatment will have been necessary, in particular in order to remove phospholipids.

The oils that are obtained are for example suitable for use in foodstuffs, in particular for child nutrition or for use as a food supplement. They can however also be used in cosmetic or pharmaceutical products.

However, the biomass that remains after pressing is a product of the process that can be used for example as animal feed, in particular for domestic pets.

It is of particular advantage here that the second biomass used acts as an additional digesting agent for the microbial biomass. If, as is normally the case, the microbial biomass is pressed alone, this normally makes very heavy demands on the pressing process. Pressing is often carried out at very high pressures and often at increased temperatures, which in turn is bad for the quality of the raw oil that is obtained, particularly in relation to possible oxidation reactions of the polyunsaturated fatty acids. If sunflower seeds are used as a second additional biomass, the husks of the sunflower seeds serve as frictional and digestion agents, and it is possible to work at reduced pressures and reduced temperatures, which considerably improves the quality of the PUFA-oil that is obtained. These effects are found even when small quantities of the second biomass are added.

Here, the oil of the oil seeds serves as a solvent for the microbial triglycerides that contain PUFAs, so that according to one preferred design example with the method according to this invention, the use of solvents to release the triglycerides can be omitted.

According to the invention, it is particularly preferable if sunflower seeds are added as the second biomass. It is however possible, and obvious to the person skilled in the art, that other second biomasses can also be used. In particular, the second biomass is a vegetable biomass, and for particular preference a biomass in the form of the fruits of plants, such as for example olives, nuts, plant leaves, plant stems and plant stalks, as well as beans, husks and the like.

For particular preference, these are the classic oil seeds: a list of the classic oil seeds can be found in textbooks that are familiar to the person skilled in the art, and need not be repeated here. As noted above, sunflower seeds are particularly preferable. The oil obtained according to this invention has a particularly low loading of phospholipids, free fatty acids, pigments, polymers and other substances, i.e. substances from the biomass that do not represent triacylglycerides.

The method of the present invention thus represents a particularly selective method for producing stable, highly purified oil that contains PUFAs. In a particularly preferred aspect, the present invention therefore relates to a method that leads to a high-quality oil that contains PUFAs, without the aggressive and cumbersome processes such as de-sliming, neutralisation, de-waxing and de-colourisation.

However, wherever it appears appropriate, the oils according to this invention are subjected to a refining step using a processing agent such as for example a silicate. Treatment with the processing agent can for example take place during filtration.

Finally, the oils are subjected to deodorisation, for example through distillation by steam entraining or through molecular distillation at a relatively low temperature. The result of this is that the resulting oil contains a particularly small proportion of trans fatty acids.

The method according to this invention also includes the use, following on from pressing, of organic solvents for normal processes such as also occur in the processing of vegetable oils, e.g. winterising, refining, bleaching, deodorisation etc. All these methods are familiar to the person skilled in the art, and do not need to be explained here for the purposes of the present invention.

Since the method is preferably carried out under a nitrogen layer and in the presence of tocopherols or tocotrienols that are either naturally present in the second biomass or are added during the process, the PUFAs that are contained are protected throughout the entire process from the harmful influence of atmospheric oxygen.

It is possible to use the biomass as a food supplement without restrictions, in particular for animal husbandry, if necessary after the removal of the solvents.

It has been shown to be particularly favourable if the two biomasses are ground together before pressing. This is particularly significant if the second biomass is an oil seed that has relatively large seeds. In the case of the preferred use of sunflower seeds as the second biomass, it has been shown that grinding of the biomasses until the remaining particle size is smaller than 250 μm was particularly favourable. In the course of this grinding, of course a portion of the triglycerides that are contained is already released in the form of oil. For grinding, it is for example possible to use a ball mill or a colloidal mill. Of particular significance here is the duration of grinding, the size of the biomass particles, the temperature during grinding, and the ratio between the quantities of the two biomasses. It is clear that since the second biomass, in the form of oil seeds, in particular sunflower seeds, has hard constituents, during grinding these as it were damage the biomass, and the oil is in part released from the biomass.

This is a particularly favourable effect, which enables particularly gentle dissolution and, at the same time, dissolution with a preliminary yield. For this reason, this grinding stage is also a particularly preferred design example of the present invention. It is preferable here if the resulting particles of the second biomass are less than 500 μm on average, preferably less than 300 μm, and for particular preference less than 200 μm. These dimensions apply to at least 90% of the biomass that was present initially.

The grinding process is continued until 90% of the biomass used has the desired particle size.

The temperature during grinding is chosen such that it lies above the melting point of the oil that is normally obtained from the second biomass. If for example the second biomass is sunflower seeds, then a temperature is chosen that lies above the melting point of sunflower oil. The temperature preferably lies at 20-80° C.

The weight ratio between the microbial biomass and the second biomass lies between 100:1 to 1:100. As explained above, the ratio of the two biomasses is quite significant in determining the PUFA concentration in the oil that is obtained. Here, no doubt ratios are preferred in which a PUFA content of between 1 and 10% is achieved in the finished designer oil, since these are concentrations that are advantageous in most pharmaceutical, cosmetic and food technology applications. If for example a biomass is obtained in which over 40% PUFAs can be obtained by pressing out the biomass, then a preferred ratio between the microbial biomass and the second biomass, in particular the oil seeds, can be selected such that the resulting oil contains about 10% PUFAs. For this, the normal yield of oil of the second biomass must be taken into consideration, which however the person skilled in the art would know at any time, without too much trouble, in the case of the conventional oil seeds.

The oil that is obtained may then need to be subjected to fine filtration, in order to remove small, insoluble particles. For this, the person skilled in the art will be familiar with many different methods; for example, the oil can be exposed to a mineral adsorbent as a filtration aid, for example silica filtration.

Finally, the filtered oil is rendered fermentation-free, wherein volatile substances are removed. This can likewise be carried out using the methods known in the prior art, although moderate conditions should be used in order not to cause any damage to the PUFAs. For example, one can use distillation by steam entraining, preferably under a vacuum, or molecular distillation.

The oil that is thus obtained can be used for example in foodstuff compositions for human nutrition just as it is, or in the form of a mixture with other oils such as for example fish oils or salad oils, or alternatively in the form of an emulsion, as salad dressing or mayonnaise. It can be a constituent of a dietetic milk for teenagers or adults, serve as infant formula for babies that are not breastfed, or as a follow-on milk for small children. It can likewise be incorporated into a pharmaceutical composition for oral, enteral or parenteral administration, or for topical, dermatological or ophthalmological use. It can also be an ingredient of a cosmetic, topical or oral composition. Finally, it can also be used as animal nutritional feed, for example as dry feed or moist feed or as milk. The remaining biomass can still be used to advantage as animal feed, and used for the purposes stated above.

In addition, extraction from the remaining biomass can take place, advantageously, using extraction with a solvent, preferably an organic solvent, and for particular preference a non-polar solvent that is not miscible with water, wherein hexane is particularly preferred, in order to be able to obtain that quantity of oil that was not released through pressing. The hexane extraction of such biomasses is described in detail in the prior art; a comprehensive description is to be found for example in EP 0 515 460 of Martek Corp.

Within the specialist field, in addition to hexane extraction a series of other extraction methods is well known, which are suitable at this point for obtaining the oil, preferably with a high triglyceride content, from the remaining press cake after pressing, e.g. extraction with chloroform/methanol or also with supercritical CO₂.

1. A method for producing an oil containing polyunsaturated fatty acids selected from the group comprising ARA, DP A, DHA, EPA, GLA, SA or DHGLA, characterised in that
a) micro-organisms are cultivated,
b) the biomass from a) is harvested,
c) the biomass that is obtained is mixed with a second biomass that is different from the biomass that is obtained from a), and
d) the oil is obtained by pressing the two biomasses from the remaining press cake.

2. A method according to claim 1, wherein after stage d), the remaining press cake is subjected to extraction with a solvent for the oil, preferably an organic solvent, and for particular preference with a non-polar solvent that is not miscible with water, wherein hexane is particularly preferred, and the oil is obtained from the solvent after extraction.

3. A method for producing an oil containing polyunsaturated fatty acids wherein the oil obtained from stage d) is mixed with an oil obtained by the method according to claim 2.

4. A method according to claim 1, characterised in that the method is carried out at a temperature that lies at least 5° C. above the melting point of the oil with the lower melting point of the oils obtainable from the two biomasses, in particular of the oil obtainable from the second biomass, and for particular preference lies between 20 and 80° C.

5. A method according to claim 1, characterised in that after stage d) the oil is refined gently, without solvents and without the use of alkaline or acid media, in that at least one of the following stages is carried out:
a) processing using silicate, and/or
b) processing using silica filtration, and/or
c) deodorisation under distillation by steam entraining.

6. A method according to claim 1, characterised in that the first biomass includes at least one biomass from one of the following microorganisms: Mortierella, Crypthecodinium (Dinoflagellates), Thraustochytrium, Schizochytrium (Labyrinthulomycetes), Phaeodactylum, Nanochloropsis, Euglena, Tetrahymena, Spirulina, Chlorella and Ulkenia.

7. A method according to claim 1, characterised in that the second biomass of stage c) is an oil seed, selected from the group comprising sunflower, rape, borage, evening primrose, linseed, black cumin, soya, palm kernels, palm, perilla, safflower, wheatgerm, maize, olives, palm kernels, sesame.

8. A method according to claim 1, characterised in that the two biomasses are ground together before the start of the pressing process.

9. A method according to claim 8, characterised in that the grinding is carried out at a temperature that is at least 5° C., above the melting point of the oil with the lower melting point of the oils obtainable from the two biomasses, in particular of the oil that is obtainable from the second biomass oil, and for particular preference lies between 20 and 80° C.

10. An oil that can be produced according to a method as claimed in claim 1.

11. An oil according to claim 10, containing no significant quantities of added organic solvents, preferably <0.2 ppm.

12. An oil according to claim 10, containing 1 to 50 percent in weight of PUFAs.

13. An oil according to claim 10, containing less than 5, percent in weight of phospholipids.

14. An oil according to claim 10, containing less than 5, percent in weight of free fatty acids.

15. A press cake, obtainable from stage d) from claim 1.

16. A phospholipid-rich composition, obtainable from the raw oil that is obtainable according to the method according to claim 1.

17. Use of the oil according to claim 10 as a food and/or food supplement.

18. Use of the oil according to claim 10 for the manufacture of a medicine for human or animal purposes.

19. Use of the oil according to claim 10 as a cosmetic for human or animal purposes.

20. Use of the press cake according to claim 15 as a food and/or food supplement for human or animal purposes, for the manufacture of a medicine for human or animal purposes and/or as a cosmetic for human or animal purposes.

21. A food according to claim 17, which is an artificial infant formula for providing complete parenteral nutrition.
